(19) United States
(12) Patent Application Publication
(10) Pub. No.: US 2004/0015201 A1
(43) Pub. Date: Jan. 22, 2004

(54) Process for electrostimulation treatment of obesity

(75) Inventor: Robert J. Greenstein, Tenafly, NJ (US)

Correspondence Address: FITCH EVEN TABIN AND FLANNERY, 120 SOUTH LA SALLE STREET, SUITE 1600, CHICAGO, IL, 60603-3406, US

(73) Assignee: Transneuronix, Inc.

(21) Appl. No.: 10/412,935

(22) Filed: Apr. 14, 2003

Related U.S. Application Data
(60) Provisional application No. 60/374,418, filed on Apr. 22, 2002.

(51) Int. Cl. A61N 1/18

(52) U.S. Cl. 607/40

(57) ABSTRACT

A process for treating obesity and/or related motor disorders by providing electrostimulation to one or more anatomical sphincters along the length of the gastrointestinal tract is provided. The present process provides improved control of obesity and other syndromes related to motor disorders of the stomach and/or gastrointestinal tract. Preferably, the present method is used in combination with electrostimulation of the stomach (preferably along the lesser curvature), and even more preferably in combination with electrostimulation of the lower or distal end of the lesser curvature (i.e., towards the pylorus) of the stomach. The process comprises artificially altering, using sequential electrical pulses for preset periods of time, the natural gastric motility of the patient to slow food transit through the digestive system.

### FIELD OF THE INVENTION

The present invention relates to an improved process using electrostimulation for treating obesity and other syndromes related to motor disorders of the stomach and/or gastrointestinal tract. The improved method of this invention provides electrostimulation to one or more anatomical sphincters along the length of the gastrointestinal tract which provides improved control of obesity and other syndromes related to motor disorders of the stomach and/or gastrointestinal tract.

### BACKGROUND OF THE INVENTION

The modern surgical orientation with regard to obesity generally entails the reduction of gastric compliance, with the aim of limiting the subject's ability to ingest food, or of reducing the food absorption surface by shortening or bypassing part of the digestive canal; both aims are sought in some surgical procedures. Until recently, surgery was the only therapy that ensures real results in patients who have exceeded obesity values close to or greater than about 40 BMI (ratio of weight to the square of the height).

All of the major surgical procedures (e.g., removal or blocking off of a portion of the stomach) currently in use have some immediate and/or delayed risks. Thus, surgery is usually considered as an extreme solution when all less invasive procedures fail. Furthermore, even surgical treatment fails in some cases, thereby requiring the surgeon to restore the original anatomical situation.

More recently, methods have been successfully employed whereby an electrostimulation device is implanted on the stomach wall. For example, U.S. Pat. No. 5,423,872 (Jun. 13, 1995) provided a process for the treatment of obesity and related disorder employing an electrostimulator or pacemaker attached to the antrum or greater curvature of the stomach. U.S. Pat. No. 5,690,691 (Nov. 25, 1997) provided a portable or implantable gastric pacemaker including multiple electrodes positionable on the inner or outer surface of an organ in the gastrointestinal tract which are individually programmed to deliver a phased electrical stimulation to pace peristaltic movement of material through the gastrointestinal tract. U.S. patent application Ser. No. 09/713,556 (filed Nov. 15, 2000) provided an improved process for treatment of morbid obesity using electrostimulation on the lesser curvature of the stomach. Although these methods have generally been successful, it is still desirable to provide improved methods for such treatments. The present invention provides such an improved process.

### SUMMARY OF THE INVENTION

The present invention provides a process for treating obesity and/or related motor disorders by providing electrostimulation to one or more anatomical sphincters along the length of the gastrointestinal tract. The present process provides improved control of obesity and other syndromes related to motor disorders of the stomach and/or gastrointestinal tract. Preferably, the present method is used in combination with electrostimulation of the stomach, and even more preferably in combination with electrostimulation of the lower or distal end of the lesser curvature (i.e., towards the pylorus) of the stomach.

The process of the present invention involves treatment of obesity and other syndromes related to motor disorders of the stomach and/or gastrointestinal tract of a patient. The process comprises artificially altering, using sequential electrical pulses for preset periods of time, the natural gastric motility of the patient to slow food transit through the digestive system.

The present invention provides a method for treatment of a motor disorder of a patient's stomach or gastrointestinal tract, said method comprising implanting an electrostimulation device comprising one or more electrostimulation leads and an electrical connector for attachment to a pulse generator such that the one or more electrostimulation leads are attached to, or adjacent to, one or more anatomical sphincters along the length of the gastrointestinal tract, whereby electrical stimulation can be provided to the one or more anatomical-sphincters through the one or more electrostimulation leads; and supplying electrical stimulation to the one or more anatomical sphincters through the one or more electrostimulation leads.

This invention also provides a method for treatment of a motor disorder of a patient's stomach or gastrointestinal tract, said method comprising implanting an electrostimulation device comprising an elongated body with a proximal and a distal end and having one or more electrostimulation leads and an electrical connector for attachment to a pulse generator at the proximal end such that the one or more electrostimulation leads are attached to, or adjacent to, one or more anatomical sphincters along the length of the gastrointestinal tract, whereby electrical stimulation can be provided to the one or more anatomical sphincters through the one or more electrostimulation leads and whereby, once the electrostimulation device is implanted, the one or more electrostimulation leads are at the distal end of the elongated body; and supplying electrical stimulation to one or more anatomical sphincters through the one or more electrostimulation leads.

The present invention also provides a method for treatment of a motor disorder of a patient's stomach or gastrointestinal tract, said method comprising implanting a first electrostimulation device comprising one or more first electrostimulation leads and a first electrical connector for attachment to a first pulse generator such that the one or more electrostimulation leads are attached to, or adjacent to, one or more anatomical sphincters along the length of the gastrointestinal tract, whereby electrical stimulation can be provided to the one or more anatomical sphincters through the one or more first electrostimulation leads; implanting a second electrostimulation device comprising one or more second electrostimulation leads and a second electrical connector for attachment to a second pulse generator such that the one or more second electrostimulation leads are attached to, or adjacent to, the stomach, whereby electrical stimulation can be provided to the stomach through the one or more second electrostimulation leads; supplying electrical stimulation to the one or more anatomical sphincters through the one or more first electrostimulation leads; and supplying electrical stimulation to the stomach through the one or more second electrostimulation leads.

### BRIEF DESCRIPTION OF THE DRAWING

FIG. 1 is a sectional view of the gastrointestinal tract showing anatomical sphincters suitable for electrostimulation using the method of the invention.

FIG. 2 is a sectional view of the stomach showing electrostimulation of the lower esophageal sphincter and the lesser curvature of the stomach.

FIG. 3 is a schematic representation of a microburst pulse train that can be applied to one or more anatomical sphincters along the length of the gastrointestinal tract and/or to the to the stomach.

### DETAILED DESCRIPTION OF THE PREFERRED EMBODIMENTS

The present invention provides a process for treating obesity and/or related motor disorders by providing an electrostimulation or pacemaker device attached to, or adjacent to, one or more anatomical spincters of the gastrointestinal tract. Suitable anatomical spincters for electrostimulation in the method of the present invention include the lower esophageal or esophago-gastric sphincter, the pyloric sphincter, and the Ileo-ceacl sphincter.

The present invention also provides a method for treating obesity and/or related motor disorders by providing a first electrostimulation or pacemaker device attached to, or adjacent to, one or more anatomical spincters of the gastrointestinal tract in combination with a second electrostimuatlion or pacemaker device attached to, or adjacent to, the stomach, and even more preferably to the lesser curvature of the stomach. Suitable anatomical spincters for electrostimulation by the first electrostimulation or pacemaker device include lower esophageal or esophago-gastric sphincter, pyloric sphincter, and the Ileo-ceacl sphincter. Even more preferably, the second electrostimulation or pacemaker device provides electrostimulation to the distal end of the lesser curvature (i.e., towards the pylorus). Preferably, the process of this invention employs stimulation of the one or more anatomical sphincters along the length of the gastrointestinal tract and/or the stomach (preferably along the lesser curvature) at a rate of about 2 to about 14 pulses/minute with each pulse lasting about 0.5 to about 4 seconds such that there is a pause of about 3 to about 30 between the pulses. More preferably, the pulse rate is about 12 pulses/minute with each pulse lasting about 2 seconds with a pause of about 3 seconds between pulses. Preferably, the pulse amplitude is about 0.5 to about 15 milliamps. More preferable, each pulse consists of a train of micro-bursts with a frequency of about 5 to about 100 sec⁻¹.

The process of the present invention involves treatment of obesity and other syndromes related to motor disorders of the stomach and/or gastrointestinal tract of a patient. The process comprises artificially altering, preferably using sequential electrical pulses for preset periods of time, the natural gastric motility of the patient to prevent or slow down stomach emptying, thereby slowing food transit through the digestive system. Placement of a first electrostimulator on one or more anatomical spincters of the gastrointestinal tract and a second electrostimular on the stomach (preferably on the lesser curvature of the stomach and even more preferably on the distal end of the lesser curvature near or adjacent to the angular notch) provides superior results as compared a single electrostimulator. The sites of electrostimulation could be phased or non-phased in relation to one another.

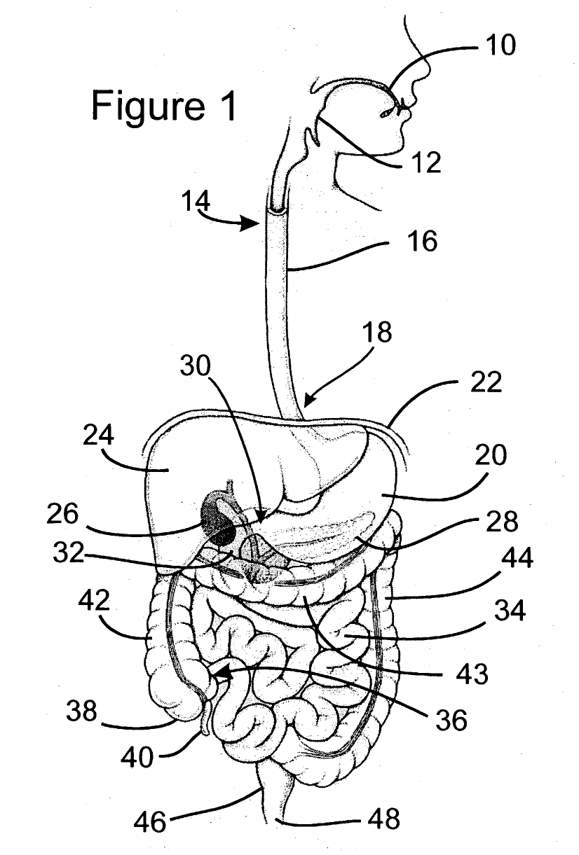

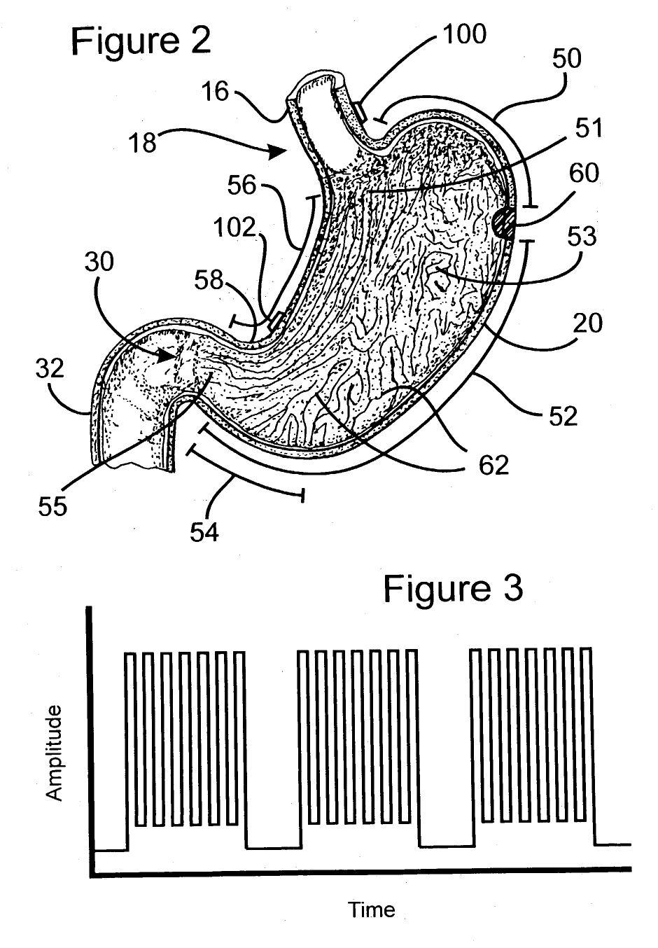

In order to further clarify the process and device for treating obesity and syndromes related to motor disorders of the stomach and/or gastrointestinal tract of a patient, according to the invention, the motor physiology of the gastrointestinal tract is briefly described. As shown in FIGS. 1 and 2, food enters the digestive tract through the mouth 10, passes by the pharynx 12, past the upper esophageal sphincter 14 into the esophagus 16, and then through the lower esophageal sphincter into the stomach 20. As shown in more detail in FIG. 2, the stomach 20 has the fundus ventriculi 50, the cardia 51, the body or corpus ventriculi 53, the greater curavature 52, the lesser curavature 56, the antrum 54, the pylorus 55, the pyloric sphincter 30, the duodenum 32, and mucous folds or rugae 62. The stomach 20 is generally divided into two parts as regards its motility: the fundus ventriculi 50, which has tonic wall movements, and the central part or corpus 53, which is characterized by phasic activity. Propulsive gastric movements begin at a point proximate to the greater curvature 53 which is not clearly identified anatomically and is termed “gastric pacemaker” 60. The gastric pacemaker 60 sends electrical pulses (depolarization potential) at a rate of approximately three times per minute which spread in an anterograde direction along the entire stomach in the form of waves which have a general sinusoidal shape. The diaphragm 22, liver 24, gall bladder 26, and pancreas 28 are also shown in FIG. 1.

The antrum 54 of the stomach 20 has a continuous phasic activity which has the purpose of mixing the food which is present in the stomach. The passage of food into the duodenum 32 is the result of a motility coordinated among the antrum 54, pylorus 55, pyloric sphincter 30, and duodenum 32. The gastric pacemaker 60 spontaneously and naturally generates sinusoidal waves along the entire stomach; these waves allow the antrum 54, in coordination with the pylorus 55, the pyloric sphincter 30, and duodenum 32, to allow food to pass into the subsequent portions of the alimentary canal (i.e., small intestines 34 and large intestines (generally consisting of ascending colon 42, transverse colon 43, and descending colon 34)). The stomach 20 releases food into the duodenum 32, the first part of the small intestines 34, where pancreatic enzymes from the pancreas 28 and bile from the liver 24 are received to aid in digestion and absorption. Food then passes through the small intestines 20 where fats and other nutrients are absorbed. After passage through the small intestines 20, the generally fluid contents pass through the Ileo-ceacl sphincter 36 into the cecum 38 with attached appendix 40. The contents then pass through the ascending colon 42, the transverse colon 43, and descending colon 34; finally, feces pass into the rectum or anal canal 46 for elimination through the anus 48.

Now that the known physiology of the gastric motility of a mammal, such as a human being, has been established, the process according to the invention consists in artificially altering, by means of sequential electrical pulses and for preset periods of time, the natural gastric motility of a patient and/or the time and manner of contraction of the lower esophageal sphincter 18, pyloric sphincter 30, and/or Ileo-ceacl sphincter 36 to prevent emptying of the stomach and/oror slow down gastric transit through the digestive system. In addition, electrostimulation of the upper portion of the gastrointestional tract may also prevent or reduce duodenal acidification during interdigestive phases and/or gastric reflux in the last portion of the esophagus 16.

More particularly, one or more of the anatomical spincters along the length of the gastrointestinal tract are subjected to electrostimulation which provides improved control of obesity and other syndromes related to motor disorders of the stomach and/or gastrointestinal tract. Suitable anatomical spincters for electrostimulation by a first electrostimulation or pacemaker device include lower esophageal or esophago-gastric sphincter, pyloric sphincter, and the Ileo-ceacl sphincter. To effect the present method, a suitable electrostimulation or pacemaker device is attached to, or adjacent to, the desired anatomical spincter. For purposes of this invention, “adjacent to” refers to a location sufficiently near the anatomical sphincter, nerves feeding the anatomical sphincter, or muscles controlling the anatomical sphincter to provide electrostimulation to the anatomical sphincter by the electrostimulation or pacemaker device. The sequential electrical pulses are generated by an electrical stimulator, such as stimulator 100 near the lower esophageal sphincter 18 in FIG. 2, which is applied by laparoscopic means. In this manner, the electrical stimulus generates one or more waves (e.g., sinusoidal waves) which modulates physiological function of the electrostimulated sphincter and associated tissue or organs. Electrostimulion of the lower esophageal or esophago-gastric sphincter would tend to retard entry of food into the stomach. Electrostimulion of the pyloric sphincter or the lleo-ceacl sphincter would tend to retard emptying of the stomach or passage of food into the large intestines, respectively.

Preferably, one or more of the anatomical sphincters and a portion of the stomach are subjected to electrostimulation. Even more preferably, electrostimulation is applied to the lesser curvature 56 of the stomach. Electrostimulation of the lesser curvature 56 adds, more or less synchronously, with the natural electrical activity of the stomach when emptying procedures are activated in the stomach. Preferably, an electrical stimulator 102 is placed on the distal end (i.e., at or near the angular notch 58) of the lesser curvature 56. The electrical stimulator induces in the stomach a motor incoordination (so-called antral tachygastria) in order to slow down or even prevent gastric transit through the pylorus into the intestine located downstream and thus allow treatment of obesity related to hyperalimentation, to modulate fasting gastric hypermotility for the treatment of relapsing duodenal ulcer in anxious subjects, and/or to improve the functionality of the lower esophageal and/or pyloric sphincters in treating reflux esophagitis and gastropathy induced by duodenogastric reflux.

The electrical stimulator or electrocatheter, according to the motor phenomenon to be corrected (e.g., induction of antral tachygastria in obesity, modulation of gastric hypermotility in anxious subjects, increase in sphincter function in reflux disorders), has a purpose-specific and potentially patient-specific frequency, intensity, duration, and period of stimulation, in addition to having a specific gastric location (i.e., on or adjacent to the anatomical sphincters) for application of the electrostimulation according to the type of disorder. The stimulator can be programmed both for continuous stimulation and for “on demand” stimulation (i.e., at the onset of a particular electrical activity which can be detected by the stimulator itself through the electrocatheter (if modified to monitor electrical activity) or under the control of the patient or medical personnel).

The electrical stimulators 100 and, if used, 102 preferably have preset operating frequencies and periods which may obviously vary according to the alteration of gastrointestinal motility to be obtained and/or to the pathological condition of the patient. Generally, the electrical stimulators 100 and, if used, 102 have operating frequencies of about 2 to about 15 pulses per minute. Preferably, the process of this invention employs stimulation at a rate of about 2 to about 14 pulses/minute with each pulse lasting about 0.5 to about 4 seconds such that there is a pause of about 3 to about 30 between the pulses. The electrical discharge of each pulse can vary from approximately 1 to 15 volts for voltage-controlled stimulation and from 2 to 15 milliamperes for constant current stimulation. More preferably, the pulse rate is about 12 pulses/minute with each pulse lasting about 2 seconds with a pause of about 3 seconds between pulses. Preferably, the pulse amplitude is about 0.5 to about 15 milliamps. More preferable, each pulse consists of a train of microbursts with a frequency of about 5 to about 100 sec⁻¹. FIG. 3 generally illustrates a microburst pulse train provided to one or more anatomical sphincters and/or the stomach (preferably along the lesser curvature).

The present invention generally uses conventional laparoscopic or minimally invasive surgical techniques to place the desired electrostimulation device 30 on, or adjacent to, the one or more anatomical sphincters or, if used, desired electrostimulation device 102 on, or adjacent to, the stomach (preferably along lesser curvature 56 and even more preferably on the distal portion of the lesser curvature (i.e., adjacent to the angular notch 58)). Conventional electrostimulation devices may be used in the practice of this invention. Such devices include, for example, those described in U.S. Pat. No. 5,423,872 (Jun. 3, 1995) (an implantable gastric electrical stimulator at the antrum area of the stomach which generates sequential electrical pulses to stimulate the entire stomach, thereby artificially altering the natural gastric motility to prevent emptying or to slow down food transit through the stomach); U.S. Pat. No. 5,690,691 (Nov. 25, 1997) (a portable or implantable gastric pacemaker employing a number of electrodes along the greater curvature of the stomach for delivering phased electrical stimulation at different locations to accelerate or attenuate peristaltic movement in the gastrointestinal tract); U.S. Pat. No. 5,836,994 (Nov. 17, 1998) (an implantable gastric stimulator which incorporates direct sensing of the intrinsic gastric electrical activity by one or more sensors of predetermined frequency bandwidth for application or cessation of stimulation based on the amount of sensed activity); U.S. Pat. No. 5,861,014 (Jan. 19, 1999) (an implantable gastric stimulator for sensing abnormal electrical activity of the gastrointestinal tract so as to provide electrical stimulation for a preset time period or for the duration of the abnormal electrical activity to treat gastric rhythm abnormalities); PCT Application Serial Number PCT/US98/10402 (filed May 21, 1998) and U.S. patent application Ser. No. 09/424,324 (filed Jan. 26, 2000) (implant device equipped with tines to help secure it in the appropriate location); U.S. Pat. No. 6,041,258 (Mar. 21, 2000) (electrostimulation device with improved handle for laparoscopic surgery); U.S. patent application Ser. No. 09/640,201 (filed Aug. 16, 2000) (electrostimulation device attachable to enteric or endo-abdominal tissue or viscera which is resistance to detachment); PCT Application Serial Number PCT/US00/09910 (filed Apr. 14, 2000; Attorney Docket Number 3581/006 PCT) entitled “Gastric Stimulator Apparatus and Method for Installing” based on U.S. Provisional Application Serial Nos. 60/129,198 and 60/129,199 (both filed Apr. 14, 1999); PCT Application Serial Number PCT/US00/10154 (filed Apr. 14, 2000; Attorney Docket Number 3581/004 PCT) entitled “Gastric Stimulator Apparatus and Method for Use” based on U.S. Provisional Application Serial No. 60/129,209 (filed Apr. 14, 1999) and 60/466,387 (filed Dec. 17, 1999); U.S. Provisional Patent Application Serial No. 60/235,660 (filed Sep. 26, 2000) entitled “Method and Apparatus for Intentional Impairment of Gastric Motility and/or Efficiency by Triggered Electrical Stimulation of the Gastric Tract with Respect to the Intrinsic Gastric Electrical Activity”; and U.S. patent application Ser. No. 09/713,556 (filed Nov. 15, 2000) entitled “Improved Process for Electrostimulation Treatment of Morbid Obesity.” All patents, patent applications, provisional patent applications, and/or publications referred to in the specification are hereby incorporated by reference.

Preferred electrostimulation devices include electrocatheters having an elongated body with a distal end having an electrostimulation lead or leads mounted on, or adjacent to, the desired anatomical sphincter and, if desired, to the stomach (preferably in the region of the lesser curvature) and a proximal end for attachment to a pulse generator. The electrostimulation lead or leads are attached to a power source through, or with, the pulse generator. Such preferred electrostimulation devices are described in, for example, PCT Application Serial Number PCT/US98/10402 (filed May 21, 1998), U.S. patent application Ser. No. 09/424,324 (filed Jan. 26, 2000), and U.S. patent application Ser. No. 09/640,201 (filed Aug. 16, 2000). If electrostimulation of both one or more anatomical sphincters and a portion of the stomach is desired, similar or different electrostimulation devices can be used in the different locations. Moreover, electrostimulation devices located at different locations within or along the gastrointestinal tract may use similar or different electrostimulation patterns and may or may not be coordinated. When two or more electrostimulation devices are used, they may have separate or combined power sources and/or pulse generators.

Although the present invention is especially adapted for treatment of obesity and/or control of weight, it may also be employed in treatment regimes involving other stomach-related disorders including, for example, relapsing peptic duodenal ulcer of anxious subjects, gastric peptic disorders induced by duodenogastric reflux, esophageal peptic disorders induced by gastroesophageal reflux, and the like.

The methods and electrostimulators used in the present invention are susceptible to numerous modifications and variations, all of which are within the scope of the present inventive concept. Furthermore, all the details may be replaced with technically equivalent elements. The materials employed, the shapes, and the dimensions of the specific electrostimulators may be varied according to the requirements.

We claim:

1. A method for treatment of a motor disorder of a patient's stomach or gastrointestinal tract, said method comprising implanting an electrostimulation device comprising one or more electrostimulation leads and an electrical connector for attachment to a pulse generator such that the one or more electrostimulation leads are attached to, or adjacent to, one or more anatomical sphincters along the length of the gastrointestinal tract, whereby electrical stimulation can be provided to the one or more anatomical sphincters through the one or more electrostimulation leads; and supplying electrical stimulation to the one or more anatomical sphincters through the one or more electrostimulation leads.

2. The method of claim 1, wherein the one or more anatomical sphincters are selected from the group consisting of the lower esophageal sphincter, the pyloric sphincter, and the Ileo-ceacl sphincter.

3. The method of claim 1, wherein the electrical stimulation supplied to the one or more anatomical sphincters has an operating frequency of about 2 to about 15 pulses per minute.

4. The method of claim 2, wherein the electrical stimulation supplied to the one or more anatomical sphincters has an operating frequency of about 2 to about 15 pulses per minute.

5. The method of claim 3, wherein the operating frequency is about 2 to about 14 pulses/minute with each pulse lasting about 0.5 to about 4 seconds such that there is a pause of about 3 to about 30 between the pulses.

6. The method of claim 4, wherein the operating frequency is about 2 to about 14 pulses/minute with each pulse lasting about 0.5 to about 4 seconds such that there is a pause of about 3 to about 30 between the pulses.

7. The method of claim 3, wherein each pulse consists of a train of micro-bursts with a frequency of about 5 to about 100 sec⁻¹.

8. The method of claim 4, wherein each pulse consists of a train of micro-bursts with a frequency of about 5 to about 100 sec⁻¹.

9. The method of claim 5, wherein each pulse consists of a train of micro-bursts with a frequency of about 5 to about 100 sec⁻¹.

10. The method of claim 6, wherein each pulse consists of a train of micro-bursts with a frequency of about 5 to about 100 sec⁻¹.

11. The method of claim 1, wherein the motor disorder is obesity.

12. The method of claim 2, wherein the motor disorder is obesity.

13. A method for treatment of a motor disorder of a patient's stomach or gastrointestinal tract, said method comprising
implanting an electrostimulation device comprising an elongated body with a proximal and a distal end and having one or more electrostimulation leads and an electrical connector for attachment to a pulse generator at the proximal end such that the one or more electrostimulation leads are attached to, or adjacent to, one or more anatomical sphincters along the length of the gastrointestinal tract, whereby electrical stimulation can be provided to the one or more anatomical sphincters through the one or more electrostimulation leads and whereby, once the electrostimulation device is implanted, the one or more electrostimulation leads are at the distal end of the elongated body; and
supplying electrical stimulation to one or more anatomical sphincters through the one or more electrostimulation leads.

14. The method of claim 13, wherein the one or more anatomical sphincters are selected from the group consisting of the lower esophageal sphincter, the pyloric sphincter, and the Ileo-ceacl sphincter.

15. The method of claim 13, wherein the electrical stimulation supplied to the one or more anatomical sphincters has an operating frequency of about 2 to about 15 pulses per minute.

16. The method of claim 14, wherein the electrical stimulation supplied to the one or more anatomical sphincters has an operating frequency of about 2 to about 15 pulses per minute.

17. The method of claim 15, wherein the operating frequency of the electrical stimulation is about 2 to about 14 pulses/minute with each pulse lasting about 0.5 to about 4 seconds such that there is a pause of about 3 to about 30 between the pulses.

18. The method of claim 16, wherein the operating frequency of the electrical stimulation is about 2 to about 14 pulses/minute with each pulse lasting about 0.5 to about 4 seconds such that there is a pause of about 3 to about 30 between the pulses.

19. The method of claim 15, wherein each pulse consists of a train of micro-bursts with a frequency of about 5 to about 100 sec⁻¹.

20. The method of claim 16, wherein each pulse consists of a train of micro-bursts with a frequency of about 5 to about 100 sec⁻¹.

21. The method of claim 17, wherein each pulse consists of a train of micro-bursts with a frequency of about 5 to about 100 sec⁻¹.

22. The method of claim 18, wherein each pulse consists of a train of micro-bursts with a frequency of about 5 to about 100 sec⁻¹.

23. The method of claim 13, wherein the motor disorder is obesity.

24. The method of claim 14, wherein the motor disorder is obesity.

25. A method for treatment of a motor disorder of a patient's stomach or gastrointestinal tract, said method comprising implanting a first electrostimulation device comprising one or more first electrostimulation leads and a first electrical connector for attachment to a first pulse generator such that the one or more electrostimulation leads are attached to, or adjacent to, one or more anatomical sphincters along the length of the gastrointestinal tract, whereby electrical stimulation can be provided to the one or more anatomical sphincters through the one or more first electrostimulation leads; implanting a second electrostimulation device comprising one or more second electrostimulation leads and a second electrical connector for attachment to a second pulse generator such that the one or more second electrostimulation leads are attached to, or adjacent to,the stomach, whereby electrical stimulation can be provided to the stomach through the one or more second electrostimulation leads; supplying electrical stimulation to the one or more anatomical sphincters through the one or more first electrostimulation leads; and supplying electrical stimulation to the stomach through the one or more second electrostimulation leads.

26. The method of claim 25, wherein the one or more anatomical sphincters are selected from the group consisting of the lower esophageal sphincter, the pyloric sphincter, and the Ileo-ceacl sphincter and wherein the one or more second electrostimulation leads are attached to, or adjacent to, the lesser curvature of the stomach.

27. The method of claim 26, wherein the the one or more second electrostimulation leads are attached to, or adjacent to, the lesser curvature at its lower end.

28. The method of claim 25, wherein the motor disorder is obesity.

29. The method of claim 26, wherein the motor disorder is obesity.

30. The method of claim 27, wherein the motor disorder is obesity.
